(19) United States
(12) Patent Application Publication
(10) Pub. No.: US 2025/0049585 A1
(43) Pub. Date: Feb. 13, 2025

(54) A MODULE-WISE POSTURE CORRECTION MASTECTOMY BRA WITH THERMAL COMFORT AND METHOD OF MAKING THEREOF

(71) Applicant: The Hong Kong Research Institute of Textiles and Apparel Limited, Hong Kong (CN)

(72) Inventors: Gloria Lei YAO, Hong Kong (CN); Hing-leung CHAN, Hong Kong (CN); Erika Kit-shan NGAN, Hong Kong (CN); Kaoru Ting-fong LEUNG, Hong Kong (CN); Kain Hung-chiu WU, Hong Kong (CN); Jasmine Li CHI, Hong Kong (CN); Yammy Yan-yi CHENG, Hong Kong (CN)

(21) Appl. No.: 18/723,631

(22) PCT Filed: Jan. 20, 2023
(86) PCT No.: PCT/CN2023/073382

§ 371 Date: Jun. 24, 2024

Related U.S. Application Data
(60) Provisional application No. 63/266,948, filed on Jan. 20, 2022.

(30) Foreign Application Priority Data
Oct. 25, 2022 (HK) 22022062661.2

(51) Int. Cl. A61F 2/52; A41C 3/00; A41C 3/14; A61F 2/50; A61F 2/76; A61F 2/78; B33Y 40/20; B33Y 50/00; B33Y 80/00

(52) U.S. Cl. CPC A61F 2/52; A41C 3/0021; A41C 3/0064; A41C 3/144; A61F 2/5044; A61F 2/76; A61F 2/78; B33Y 40/20; B33Y 50/00; B33Y 80/00; A61F 2002/7685; A61F 2002/7862

(57) ABSTRACT

The present disclosure provides a customized module-wise bra with both aesthetic, comfort, and functions assisting a post-mastectomy subject to wear the bra and correct any postural distortion arising from imbalance due to loss of weight from the mastectomized breast. Also provided herein is a method for fabricating the customized module-wise bra according to the anatomical/physical characteristics of the subject based on various 3D printing, 3D body scanning and ultrasonography techniques.

### CROSS-REFERENCE TO RELATED APPLICATIONS

This application claims priorities from (1) the U.S. provisional patent application Ser. No. 63/266,948 filed Jan. 20, 2022; and (2) the Hong Kong patent application number 22022062661.2 filed Oct. 25, 2022, the disclosures of which are incorporated herein by reference in their entirety.

### TECHNICAL FIELD

The present disclosure generally relates to ultrasonography and three-dimensional (3D) scanning for breast's mass density and volume estimation, 3D printing or additive manufacturing for breast prostheses fabrication, assembly of a module-wise thermal comfort, posture correction mastectomy bra.

### BACKGROUND

According to the World Health Organization (WHO), in 2020, there were 2.3 million women diagnosed with breast cancer and 685,000 deaths globally. Treatment of breast cancer includes surgical removal, radiation therapy and medication (hormonal therapy, chemotherapy and/or targeted biological therapy). When cancers are large, mastectomy (complete removal of the breast) may be required.

Mastectomy, either unilateral or bilateral, disturbs the distribution of body mass, and mastectomized women may experience muscle imbalance and restricted body movement. As a result, the bodies of mastectomized women will rearrange or shift to maintain body balance. It induces scoliosis, forward leaning, and shoulder and neck asymmetry. Although surgery such as breast reconstruction can restore balance between the two breasts, many mastectomized women are unable to undergo breast reconstruction because of a number of reasons, e.g. not willing/suitable to have additional surgery, financial concern, etc.

Mentally, mastectomy has a deep and negative impact on a woman because she may feel loss of femininity, fertility, charm and sexuality, fear of recurrence and change of family role. Wearing external breast prosthesis, also called breast form, is an alternative to restore balance and also gain confidence. Physically, external breast prosthesis has a positive effect on muscle tone distribution, and hence restore the posture balance. Also, the use of an external breast prosthesis has the advantages of aesthetic function, provide the missing weight and volume according to the body structure, and look balanced without surgery, e.g. breast reconstruction.

In long term consideration, as the breast density is decreasing during and after menopause, in order to match the weight of external breast prosthesis and the existing breast of unilateral mastectomized women, regular estimation of breast density is necessary. In addition, the weight of the external breast prosthesis shall be adjusted with the consideration of the user's feeling whether she thinks it is too heavy or too light.

On the market, two common types of external breast prosthesis can be found, which are conventional non-adhesive external prosthesis and adhesive external prosthesis. In general, materials for making external breast prosthesis include silicone, foam or polyfill, rubber- or cotton-filled cloth bags, and plastic pellets- or seed-filled cloth bags. Nowadays, 3D printed personalized prosthetic breasts are also available but limited to few countries only. However, thermal discomfort is still a major dissatisfaction. Also, high price is a concern for the lower class.

Previous 3D printed external breast prostheses, such as that described in WO 2019/164390 A1, use inner walls perpendicular to chest wall and filling of gel pads, filled silicone bags of air. The gel pads or bags of filling are not permeable to water vapour, hindering the evaporation of sweat for thermal regulation.

### SUMMARY OF INVENTION

Accordingly, the present disclosure proposes an external breast prosthesis or bra pad with thermal comfort whilst posture correction function for a user thereof.

In a first aspect, there is provided a breathable, weight counterbalancing breast prosthesis comprising a bra pad module comprising a 3D printed bra pad enclosing at least a weight insert customized to a user thereof.

A second aspect of the present invention provides a module-wise mastectomy bra comprising basic bra components and customized structural and functional features according to the needs of a specific user, where the specific user features a unilateral mastectomy, or a bilateral mastectomy provided a database of women's breast anatomy information of corresponding community is available.

A third aspect of the present invention provides a method of making a customized module-wise mastectomy bra for a specific user.

In certain embodiments, the customized structural and function features of the module-wise mastectomy bra include, but not limited to, one or more of reinforcement band and sling, chest panel, and pressure release shoulder strap.

In certain embodiments, the basic bra components include, but not limited to, cups, centre gore, underband wings, shoulder straps, and a release/closing mechanism.

In certain embodiments, the method for making the customized module-wise mastectomy bra includes estimating breast volume by 3D body scanning, estimating breast mass density by ultrasonography with 3D body scanning for unilateral mastectomized subjects as well as bilateral mastectomized subjects (with the database support), selecting materials and weight for the weight insert, 3D printing bra pad of the mastectomy bra, customizing structural and functional features according to anatomical and/or physical characteristics of the user such as body mass index (BMI), assembling the 3D printed bra pad and the weight insert into the bra pad module together with a mastectomy bra template incorporated with the corresponding customized structural and functional features specific to the user to form the module-wise mastectomy bra of the present invention.

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Other aspects of the present invention are disclosed as illustrated by the embodiments hereinafter.

### BRIEF DESCRIPTION OF DRAWINGS

The appended drawings, where like reference numerals refer to identical or functionally similar elements, contain figures of certain embodiments to further illustrate and clarify the above and other aspects, advantages and features of the present invention. It will be appreciated that these drawings depict embodiments of the invention and are not intended to limit its scope. The invention will be described and explained with additional specificity and detail through the use of the accompanying drawings in which:

FIG. 1 shows images from multiple views of a bra pad module prepared according to an embodiment of the present invention: back view (left), front view (middle), and side view (right);

FIG. 2 shows images from different views of a weight insert for the bra pad module prepared according to an embodiment of the present invention: top view (upper), side view (bottom left), front view (bottom right); the dotted circle with a cross indicates the centre of the weight insert in this embodiment;

FIG. 3 shows an image of a fabric bag enclosing the 3D printed bra pad and the weight insert assembly;

FIG. 4 shows images of implementing 3D scanning step of the present method according to an embodiment of the present invention;

FIGS. 5A-K schematically depict a conventional method of assembling a module-wise mastectomy bra from different components: FIG. 5A is a perspective front view indicating basic bra components including cups, cradle, wings, plastic bones, shoulder straps, underband, and front closure; FIG. 5B is a back view indicating back closure (in close state) and hidden pocket; FIG. 5C is a perspective back view where the back closure is in open state; FIG. 5D is a perspective front view where a chest panel is incorporated; FIG. 5E is a perspective front view where a pressure release shoulder strap is incorporated; FIGS. 5F to 5K are back view or perspective view showing different embodiments of reinforcement band and sling;

FIGS. 6A-H schematically depicts various embodiments of customizing reinforcement band and sling for the present customized module-wise mastectomy bra to exert different forces or support (indicated by arrows) towards the bra pad module and various parts of the wearer's body: FIG. 6A shows a non-stretchable fabric on cradle and center gore; FIG. 6B shows a four-way stretchable fabric on front panel and a two-way stretchable fabric on shoulder strap; FIG. 6C shows a four-way stretchable fabric partially covering the front panel; FIG. 6D shows another four-way stretchable fabric partially covering the front panel; FIG. 6E shows a two-way stretchable fabric attached to the neckline panel and the underarm plastic bones; FIG. 6F shows a two-way stretchable fabric attached to the underarm panel and the cradle; FIG. 6G shows a two-way stretchable fabric on cradle and wing; FIG. 6H shows a four-way stretchable fabric at back panel;

FIG. 7 illustrates the locations of breast scanned by ultrasonography to estimate mass density according to certain embodiments of the present invention;

FIG. 8 is a flow chart showing process steps of making the customized module-wise mastectomy bra according to certain embodiments of the present invention.

Skilled artisans will appreciate that elements in the figures are illustrated for simplicity and clarity and have not necessarily been depicted to scale;

FIG. 9 is a flow chart summarizing different stages of customized mastectomy bra preparation according to certain embodiments of the present invention.

### DETAILED DESCRIPTION OF THE INVENTION

It will be apparent to those skilled in the art that modifications, including additions and/or substitutions, may be made without departing from the scope and spirit of the invention. Specific details may be omitted so as not to obscure the invention; however, the disclosure is written to enable one skilled in the art to practice the teachings herein without undue experimentation.

The present disclosure provides a low-cost, module-wise, time-saving, and customizable mastectomy bra with sufficient thermal comfort and body balance improvement. In accordance with the wearer's existing breast contour, volume and mass-density data obtained by the present method according to certain embodiments, a customized module-wise mastectomy bra can be made with structural and functional features to facilitate the use of the mastectomy bra by the particular wearer.

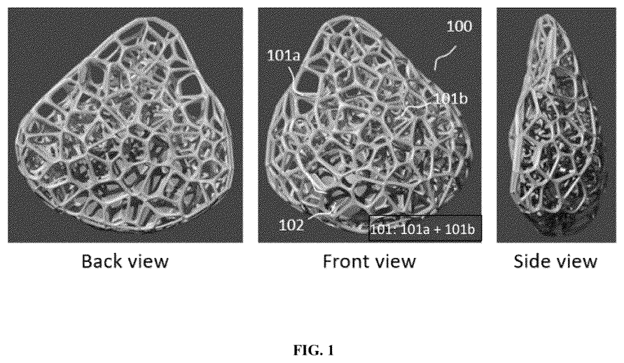

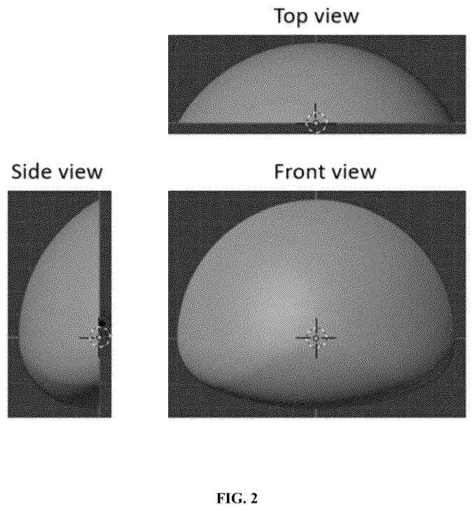

FIG. 1 provides a bra pad module 100 prepared by the present method of making a customized module-wise mastectomy bra, including a bra pad 101 which consists of a layer of lower density 101a representing the fat tissue and a layer of higher density 101b representing the fibroglandular tissue. The bra pad according to exemplary embodiments of the present invention is made of one or more elastic materials and configured to be multi-directionally compressible. To enable these functionalities, the present bra pad is 3D printed. Unlike some conventional 3D printing methods for making three-dimensional internal architecture such as the one disclosed in WO 2019/175901 A1 which results in a continuous internal wall-like structure perpendicular to the chest wall that creates stiffness in the grid along the direction, the present bra pad according to the exemplary embodiments is modelled by a Voronoi-like 3D print grid system which is built according to the wearer's existing breast density. The Voronoi-like grid model is designed by randomly dividing a 3D breast model volume into numerous irregular unit cells, where each of the cells in a Voronoi-like grid has a seed point. Connections (or connection lines) among neighboring cells are always at halfway of each pair of neighboring cells. Since the randomly divided cells are built on volume basis, no continuous internal wall perpendicular to the chest wall is required. Stresses within the bra pad grid can therefore be equally distributed.

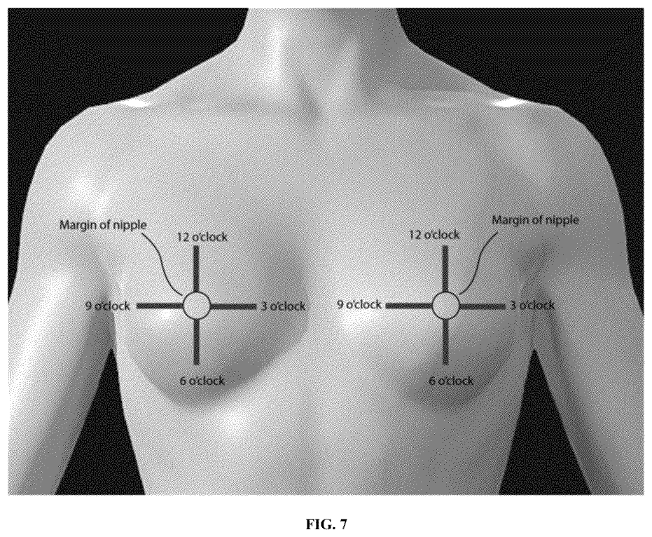

As seen in FIG. 7, the 3D printed bra pad 101 can be divided into four quadrants, being upper, right, lower and left quadrants, corresponding to ultrasound scans by ultrasonography at positions of 12, 3, 6 and 9 o'clock, respectively. In exemplary embodiments, printing density of the grid depends on corresponding ultrasonography data. In other words, quadrant with higher proportion of fibroglandular tissue will be 3D printed with higher density to resemble the tissue distribution of the existing breast.

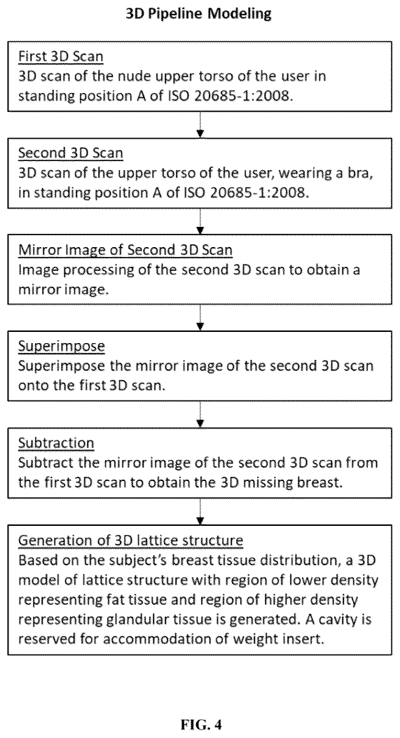

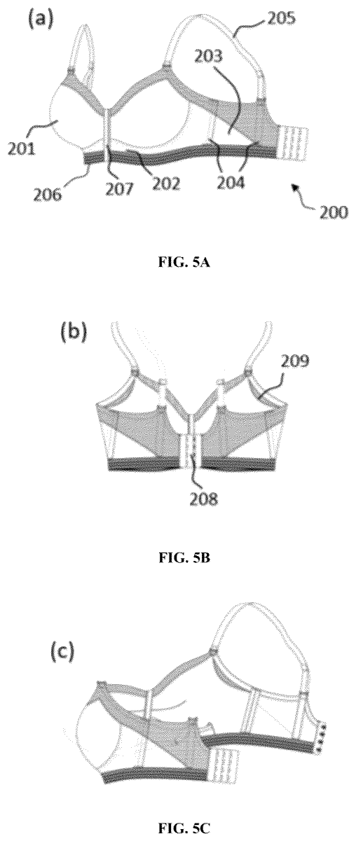

In addition to mechanical properties, the void spaces in the Voronoi-like grid system are interconnected, such that water vapour from sweat evaporation can penetrate the grid and subsequently disperse into the surroundings in multiple directions, through which the body heat is also dissipated to the surroundings, exerting a moisture management function and breathability. FIG. 4 depicts a 3D pipeline modelling for generating the size, Voronoi structure, shape and volume of the printed bra pad customized for a particular wearer. The 3D printed bra pad 101 according to the Voronoi-like grid system inserted with a carefully selected weight insert 102 is enclosed into a fabric bag 103 (an embodiment of the fabric bag is shown in FIG. 3) to form the bra pad module 100. In certain embodiments, the material for the fabric bag is also selected with good moisture management and breathability, in addition to the material of the bra pad 101 or weight insert 102.

In certain embodiments, the bra pad module 100 is from about 40 wt. % to 60 wt. % of the existing breast of the user. The weight of the bra pad 101 built under the Voronoi-like grid structure is tunable by increasing the number of void space in order to reduce the weight of the bra pad in the case where a denser (or heavier) weight insert based on the ultrasound scans is required, or vice versa. Normally, the weight of the bra pad may not reach 50% of the missing breast. The selection of weight insert should also take this weight limit into account.

In certain embodiments, the density of elastic material(s) making up the weight insert is higher than 1 g/ml.

In certain embodiments, the one or more elastic materials can be one or more of thermoplastic elastomer (TPE), thermoplastic polyurethanes (TPU), resin, silicone, and viscoelastic gel.

It is generally understood that the more void space the bra pad has, the more breathable is the overall bra pad module.

In certain embodiments, the 3D printed bra pad is coated by a durable water repellent layer selected from the group consisting of silicon-based, wax-based, and plant-based polyfluorocarbon (PFC)-free materials, or any combination thereof.

The 3D printed bra pad based on the 3D scanned images has an outer surface and a base matching the contour of the existing breast and the scar area of the missing breast, respectively.

The position of the empty space in the bra pad 101 for accommodating the weight insert 102 is determined according to the wearer's morphological features and/or physical parameters including, but not limited to, body mass index (BMI). In certain embodiments, the position of the empty space is configured to be about half or one-third height of the existing breast. Other factors that need to be considered when positioning the empty space thereof include centre of gravity with respect to the overall weight of the bra pad module.

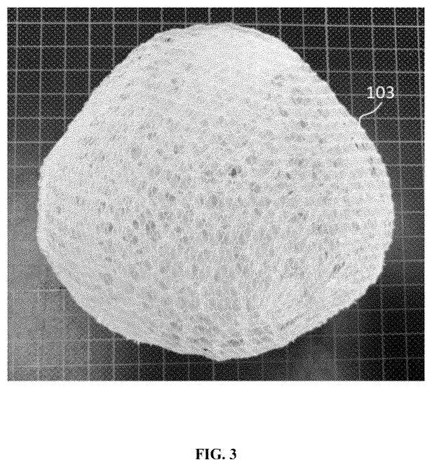

FIG. 2 illustrates an example of weight insert prepared according to certain embodiments of the present invention mainly based on the mass density evaluation by ultrasonography of the existing breast. In exemplary embodiments, the weight and proportion of fat to fibroglandular tissue of the existing breast of a unilateral mastectomized subject are determined prior to the selection of material for the weight insert or standard weight insert from a collection of standardized weight inserts. Based on the 3D volume of the existing breast scanned by 3D body scanning, the density of the existing breast in terms of the fat and fibroglandular tissue can be determined by the following equation with the mass estimated by the ultrasound scans:

$$Density = \frac{Mass}{Volume} \qquad ( 1 )$$

The following table (Table 1) summarizes mass density of fat and fibroglandular tissues from a literature (Sanchez, A., et al, 2017):

TABLE 1

| Type of Tissue | Mass density (g/ml) |
|---|---|
| Fat | 0.900 |
| Fibroglandular | 1.057 |

Taking the above literature mass density for each type of tissue as reference, the breast mass density of the existing breast can be determined by the following equation:

$${ρ}_{B} = A {ρ}_{F} + ( 1 - A ) {ρ}_{G} \qquad ( 2 )$$

where the breast is assumed to be composed entirely of fat and fibroglandular tissues, in which ρ_{F} denotes the mass density of fat tissue; ρ_{G} denotes the mass density of fibroglandular tissue; A denotes proportion of fat in the breast.

FIG. 4 provides an embodiment of how the volume of the missing breast in a unilateral mastectomized subject is determined, which is by 3D scanning method. In accordance with a well-established 3D scanning method disclosed in US 2017/0281367 A1, which is incorporated herein by reference, initially a nude upper torso section of the wearer is 3D scanned, followed by a 3D scan of the upper torso section of the wearer in the same position covered with a bra at this time, generating a mirror image of the scanned bra from the subsequent 3D scan, reconstructing the 3D shape of the missing breast of the mastectomized side of the wearer based on the difference between a mirror image of the initial 3D scan and the mirror image of the subsequent 3D scan of the upper torso section of the wearer such that a 3D shape of the missing breast can be mapped out, thereby determining the volume of the 3D missing breast.

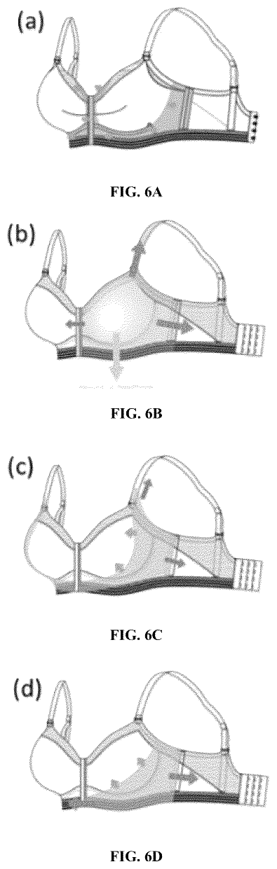

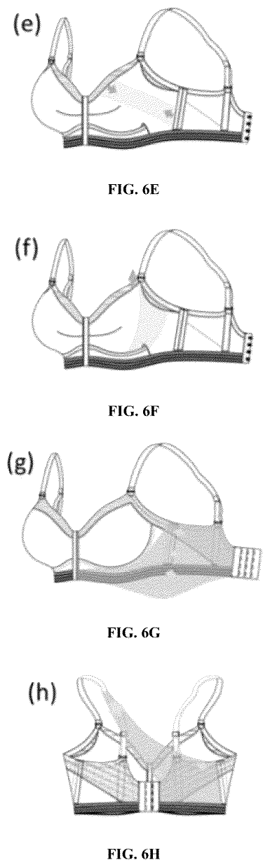

As described hereinabove, the size of the bra pad of the bra pad module for the missing breast of the wearer is determined by a 3D pipeline modeling based on random Voronoi approach, according to the embodiments depicted in FIG. 6. The Voronoi-like 3D printed grid system as such enables the bra pad to deform multi-directionally and move alongside the wearer's motion. Corresponding weight insert is provided in a designated position in the bra pad to suit the anatomical/physical features of the wearer's remaining tissues after mastectomy while counteracting center of gravity of the bra pad module.

In certain embodiments, the initial (first) 3D scan of the wearer's nude upper torso complies with the standard of ISO 20685-1:2018, in which the wearer stands in position A according to said standard during 3D scanning with the upper torso thereof exposed.

In certain embodiments, the subsequent (second) 3D scan of the wearer's upper torso also complies with the standard of ISO 20685-1:2018 in which the wearer stands in position A according to said standard during 3D scanning without exposing the upper torso thereof by covering the same with a bra.

The volume of the missing breast can be determined by subtracting the mirror image of the subsequent 3D scan of the covered upper torso by the first 3D scan of the nude upper torso.

In certain embodiments, the centre of gravity of the bra pad module is adjustable by adjusting the weight of the weight insert.

In certain embodiments, the weight of the weight insert corresponds to the estimated weight of the wearer's existing breast.

In certain embodiments, the weight insert can be some standardized weight insert or tailor-made.

The following table (Table 2) provides an example of some standardized sizes and their corresponding mass of possible weight inserts:

TABLE 2
Example of mass of Standardized Weight Inserts

| Size | Mass of Weight Insert (g) |
|---|---|
| S | 100 |
| M | 150 |
| L | 200 |
| XL | 250 |
| XXL | 300 |
| XXXL | 350 |

Apart from those provided in Table 2, other sizes can be included by extrapolation either up or down the size range.

Preferably, the overall weight of the bra pad module is about 40-60 wt. % of the missing breast.

Similar to the library of breast volume, density and shape for silicone prosthesis, the bra pad of the present invention can be selected from a library of ready-made 3D printed bra pads with different volume, density, shape and even breathability in terms of moisture and other elements affecting the thermal comfort of the wearer.

Once the bra pad 101 is 3D printed, and a corresponding weight insert 102 is selected, they are put together in an enclosure, e.g., a fabric bag, to form a bra pad module 100 in order to be assembled and secured on a ready-made mastectomized bra 200, where the mastectomized bra 200 is configured to have a means for accommodating the bra pad module 100, e.g., a pocket of a bra, or a fastening mechanism, e.g., button, hook and loop fastener.

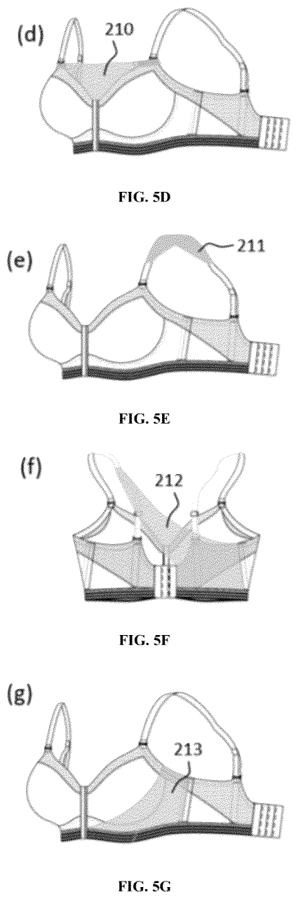

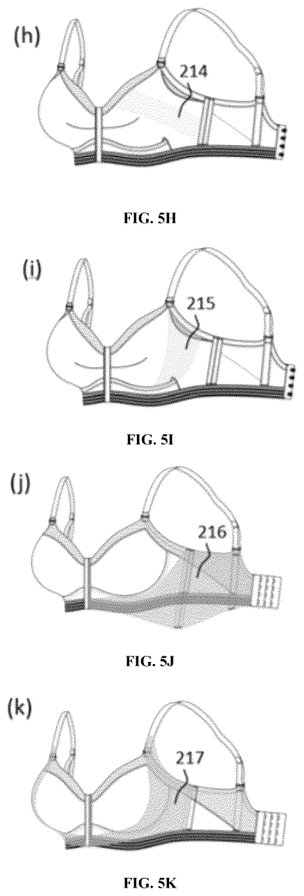

Turning to FIGS. 5A-5C, an embodiment of the present module-wise posture correction bra 200 is depicted, which includes basic bra components such as cups 201, cradle 202, wings 203, plastic bones 204, shoulder straps 205, underband 206, front closure 207, and back closure 208 (not seen from the perspective front view in FIG. 5A). In certain embodiments, a special feature, hidden pocket 209 (also not seen from the perspective front view in FIG. 5A), is disposed at the cup(s) where the breast(s) is/are missing and configured to accommodate the bra pad module 100 described herein. Other features such as some structural and functional elements to aid a mastectomized subject to circumvent difficulties in wearing and/or securing the bra arising from or caused by the mastectomy, e.g., reduced range of arm movement after mastectomy. In those circumstances, a usual fastening mechanism, e.g., back closure 208 as shown in FIGS. 5B and 5C, is hard for those mastectomized subject with difficulties in arm movement to fasten it. Therefore, in certain embodiments, the present module-wise posture correction bra can be configured with either or both of front and back fastening mechanisms. Other fastening mechanisms not described hereinabove can also be used in the present invention, so long as the wearer can operate the present bra without undue difficulties.

As seen in FIGS. 5D-5K, optional elements such as a chest panel (or cover up) 210, a pressure release shoulder strap 211, different asymmetrically arranged reinforcement bands and slings (212, 213, 214, 215, 216, 217) can be incorporated into the present bra to facilitate the wearer to wear the bra, enhance the comfort and/or correct certain postures. For example, a reinforcement band 212 as shown in FIG. 5F joins the right cradle and left shoulder strap across the back of the bra to provide a retraction force on the right upper back. Depending on the corresponding postural distortion of the wearer, different reinforcement bands and slings are intended to apply retraction force on the body of the wearer at specific target region.

FIGS. 6A-6H depicts different embodiments of customizing the present mastectomy bra by incorporating one or more optional elements such as bands and slings at different positions of the bra, in addition to the bra pad module described herein, in which FIG. 6A shows a non-stretchable fabric on cradle and center gore forming a relatively rigid structure to maintain the shape of bra; FIG. 6B shows a four-way stretchable fabric on front panel and a two-way stretchable fabric on shoulder strap pressing the bra pad module against the chest; FIG. 6C shows that a four-way stretchable fabric partially covers the front panel pushing the bra pad module and the existing breast towards the center of chest; FIG. 6D shows that a four-way stretchable fabric partially cover the front panel providing a upward support for the bra pad module; FIG. 6E shows a two-way stretchable fabric attached to the neckline panel and the underarm plastic bones pressing the bra pad module against the chest; FIG. 6F shows a two-way stretchable fabric attached to the underarm panel and the cradle pressing the bra pad module against the chest; FIG. 6G shows a two-way stretchable fabric on cradle and wing pressing the body towards the opposite side; and FIG. 6H shows a four-way stretchable fabric at back panel pressing the body towards the opposite side.

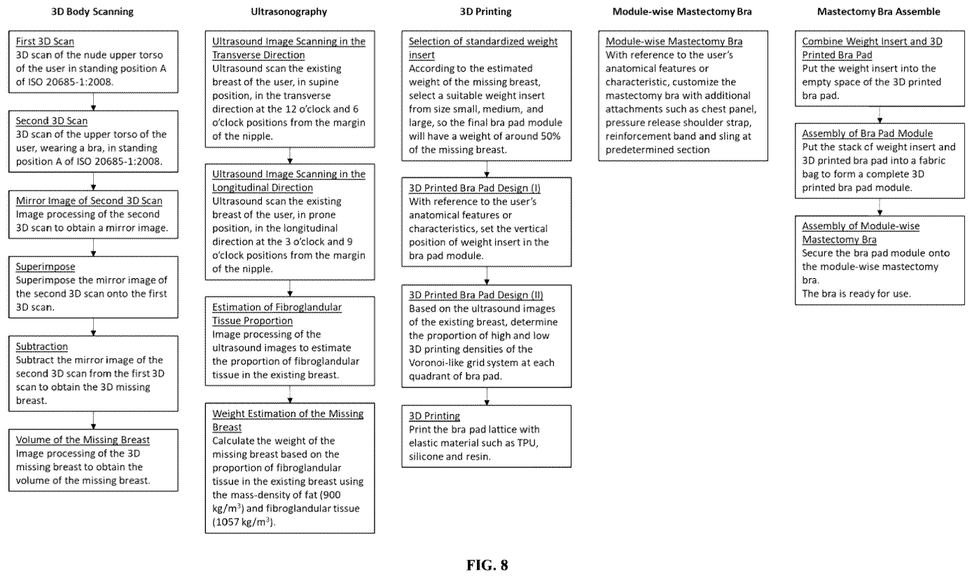

Detailed workflow of each of the steps of fabricating a customized posture correction mastectomy bra according to certain embodiments is depicted in FIG. 8. In FIG. 8, the first column from the left shows a workflow of the 3D body scanning for estimating mainly the size and volume of the missing breast from the existing breast by a serial scanning of the upper torso of the wearer in a standing position A according to ISO 20685-1:2008. A nude upper torso of the wearer (user) is first scanned, followed by a second scan of the same portion of the wearer's body in the same standing position but in the presence of a standard bra covering the breast part. By using a computer processor, a mirror image is generated from the second 3D scan. The mirrored 3D scanned image of the second scan will be superimposed with the 3D scanned image of the first scan. The 3D contour (shape) of the missing breast is mapped out by subtracting the mirror image of the second 3D scan from the image of the first 3D scan.

Turning to the second column from the left of FIG. 8, a workflow of determining the breast mass density of the existing breast is depicted, which is performed by scanning the existing breast in both transverse and longitudinal directions across the nipple position by ultrasonography. The wearer (user) will first change to a supine position during ultrasound scanning. The existing breast of a unilateral mastectomized wearer is subjected to the ultrasound scanning in the transverse direction at 12 and 6 o'clock positions from the margin of the nipple, as depicted in FIG. 7. Similarly, but at this time the wearer will be changed to a prone position during ultrasound scanning, the breast mass density of the existing breast is scanned in the longitudinal direction at 3 and 9 o'clock positions from the margin of the nipple.

The obtained ultrasound images from the ultrasonographic scanning of the existing breast of the wearer are fed to a computer processor to estimate the proportion of fibroglandular tissue, given the assumption provided in equation (2) as described herein that the breast is composed entirely of fat and fibroglandular tissues. The weight of the missing breast is thereby calculated based on the proportion of fibrogladular tissues estimated in the existing breast and subject to the standard mass density of fat and fibroglandular tissues (900 and 1057 kg/m³, respectively, from Table 1 referenced to Sanchez, A., et al, 2017). It should be noted that different ages, races, prodigies, etc. may result in different ratio of fat to fibroglandular tissues in a breast. A person of the ordinary skill should acknowledge that this ratio is substitutable if needed when calculating the mass density of different tissues in a breast of an individual.

Turning to the middle column of FIG. 8, a suitable weight insert is selected from a collection of standardized weight insert for bra, e.g., the weight inserts in different sizes and their corresponding weight as shown in Table 2. In certain embodiments, the weight insert is tailor-made instead of being selected from a collection of standardized weight insert. The main objective of selecting or making a suitable weight insert is to make the final weight of the bra pad module (such as the bra pad module 100 shown in FIG. 1) be about 40 wt. % to 60 wt. % of the existing breast (or the estimated weight of the missing breast). Preferably, the final bra pad module is about 50 wt. % of the existing (or missing breast).

Based on the 3D body scan data and ultrasound image data obtained according to the two workflows depicted in the first and second columns of FIG. 8, a 3D bra pad in grid structure (lattice form) is fabricated. In exemplary embodiments, the bra pad is constructed with at least an outer surface and a base conforming the contour of the existing breast and that of the scar area of the missing breast, respectively, based on the 3D body scan data. With reference to the wearer's anatomical/physical features obtained from the 3D body scan, it provides a vertical position of the weight insert disposed in the bra pad module, which is preferably about half to one-third of the height of the breast measured from the bottom of the breast/bra edge. Since a random Voronoi approach is employed to generate the grid structure, the densities of the randomly distributed unit cells in the grid depend on the intensity of the signal from the ultrasound images obtained by ultrasonography detected in different quadrants assigned according to the scheme as shown in FIG. 7. The Voronoi-like grid structure as-generated possesses a multi-directional compressibility, good moisture management properties, air breathability, and sufficient elasticity (Young's modulus) while mechanical strengths are not compromised. In addition, numerous void spaces in the Voronoi-like grid structure defined by the grid structure are interconnected to enable water vapor together with body heat energy be easily evaporated and dissipated to the surroundings through the grid. An exemplary material for forming the Voronoi-like gird structure of the present invention is selected from one or more of thermoplastic elastomer (TPE), thermoplastic polyurethane (TPU), silicone, resin and viscoelastic gel.

In certain embodiments, a 3D pipeline modeling such as that depicted in FIG. 4 is employed to generate size, shape, volume, and Voronoi-like grid structure of the 3D printed bra pad customized for a particular wearer.

Turning to the second column from the right of FIG. 8, a module-wise mastectomy bra incorporated with one or more structural and functional elements and other additional attachments such as chest panel, pressure release shoulder strap, reinforcement bands and slings at different predetermined section of the bra according to the wearer's anatomical/physical features and/or needs, e.g., correcting a particular postural distortion arising from imbalance between left and right sides of the breast region post-mastectomy. Some embodiments of incorporating different elements and additional attachments, apart from the bra pad module and its corresponding engaging mechanism, into the module-wise mastectomy bra are depicted in FIGS. 5D-5K and 6A-6H, respectively.

Turning to the first column from the right of FIG. 8, a workflow of assembling the bra pad, selected weight insert, and enclosure into the bra pad module and securing the bra pad module to the module-wise mastectomy bra is depicted. Initially, the selected weight insert is placed at a predetermined position in the accommodation space of the 3D printed bra pad to form a stack. The stack of the weight insert and 3D printed bra pad will be enclosed in an air breathable and water permeable bag to form an assembly. Desirable material for making the weight insert and the enclosure of the stack of weight insert and 3D printed bra pad includes any material that is air breathable, water permeable, and does not cause irritation or release harmful substances towards the skin of the wearer. The assembly as-formed is the bra pad module. Optionally, the bra pad can be coated with a water repellent layer prior to the assembly. One or more materials selected for forming the water repellent layer include silicon-based, wax-based, and plant-based polyfluorocarbon (PFC)-free materials, or any combination thereof. To secure the bra pad module to the module-wise mastectomy bra, the module-wise mastectomy bra is preferably incorporated with a pocket for receiving the bra pad module or an engaging mechanism including, but not limited to, a button, a hook and loop fastener, or any feasible fastening means. After securing the bra pad module, the structural and functional elements, and other additional features customized for the particular wearer, a customized module-wise mastectomy bra is ready for use.

Components assembled or incorporated into the present invention as described herein are mostly easily replaceable, washable, reproducible and modifiable, so that the module-wise mastectomy bra as such can be used for a longer term and suit the needs of the wearer from time to time.

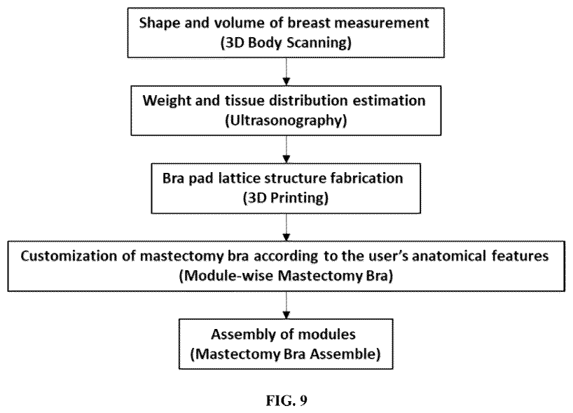

FIG. 9 summarizes the key steps/stages of preparing a personalized, module-wise posture correction mastectomy bra according to certain embodiments of the present invention.

Although the invention has been described in terms of certain embodiments, other embodiments apparent to those of ordinary skill in the art are also within the scope of this invention. Accordingly, the scope of the invention is intended to be defined only by the claims which follow.

### REFERENCES

The following references are cited or referred to in various parts of the present disclosure:
1. https://www.who.int/news-room/fact-sheets/detail/breast-cancer
2. International Patent Application Publication No. WO 2019/164390 A1, Munoz Arellano, E. A. (2019). “Custom-made external breast prosthesis”
3. Kim, W. H., et al., Ultrasonographic assessment of breast density. Breast Cancer Res. Treat., 2013, 138:851-859.
4. Sanchez, A., et al., Estimating breast mass-density: a retrospective analysis of radiological data. The Breast Journal, 2017, 23 (2): 237-239.
5. U.S. Patent Application Publication No. US 2017/0281367 A1. Ketchum, M. J. (2017). “3D-printed unibody mesh structures for breast prosthesis and methods of making same”
6. ISO 20685-1:2018 3-D scanning methodologies for internationally compatible anthropometric databases-Part 1: Evaluation protocol for body dimensions extracted from 3-D body scans.
7. Hojan, K., Ozga-Majchrzak, O., and Manikowska, F., The weight of external breast prosthesis as a factor for body balance in post-mastectomy women. Archives of Physical Medicine and Rehabilitation, 2017, 98 (10): e121-e121, Research Poster 310002.
8. Angelucci, G., and Mollaioli, F., Voronoi-like grid systems for tall buildings. Frontiers in Built Environment, 2018, 4, 78.
9. International Patent Application Publication No. WO 2019/175901 A1, Garg, V. (2019). “System and method of manufacturing prostheses”
10. Kelemen, L. E., et al., Age-specific trends in mammographic density: the Minnesota Breast Cancer Family Study. American Journal of Epidemiology, 2008, 167 (9): 1027-1036.

What is claimed is:

1. A customized module-wise posture correction bra for a mastectomized subject, comprising:
a bra pad module customized for the mastectomized subject; and
one or more structural and functional elements for facilitating wearing of the bra by the mastectomized subject, supporting the bra pad module, and/or correcting any postural distortion of the mastectomized subject post-mastectomy,
the bra pad module comprising an enclosure housing a 3D printed bra pad comprising a space for accommodating a weight insert and a plurality of voids defined by a grid structure, the grid structure being configured to be multi-directionally compressible and movable alongside the mastectomized subject's motion;
the weight insert being disposed within the space of the 3D printed bra pad in accordance with the mastectomized subject's anatomical and/or physical features and the centre of gravity of the bra pad module.

2. The customized module-wise posture correction bra of claim 1, wherein the bra pad module has a weight of about 40% to 60% of an existing breast of the mastectomized subject, and the mastectomized subject is a unilateral mastectomized.

3. The customized module-wise posture correction bra of claim 1, wherein the bra pad module weight is determined with reference to a database of women's breast anatomy information of a corresponding community when the mastectomized subject is a bilateral mastectomized subject.

4. The customized module-wise posture correction bra of claim 1, wherein the bra pad module has a proximal surface to chest wall of the mastectomized subject with a contour substantially in conformity with the chest wall's contour.

5. The customized module-wise posture correction bra of claim 1, wherein the grid structure defining the plurality of voids is a 3D printed grid based on Voronoi-like grid model such that a number of randomly distributed, irregular unit cells is formed.

6. The customized module-wise posture correction bra of claim 4, wherein each of the irregular unit cells has a seed point such that connections between each pair of neighboring unit cells are configured to be halfway of the grid line between the two neighboring cells.

7. The customized module-wise posture correction bra of claim 2, wherein the weight insert is either custom-made or selected from a collection of standardized weight inserts having a fixed value or range of mass for each of the standard sizes corresponding to an estimated mass density of fat and fibrograndular tissues of the existing breast.

8. The customized module-wise posture correction bra of claim 1, wherein the enclosure of the bra pad module is selected from a fabric bag made of air-breathable and water-permeable material.

9. The customized module-wise posture correction bra of claim 1, wherein the one or more structural and functional elements comprise reinforcement bands and slings disposed around different parts of the bra for facilitating wearing of the bra by the mastectomized subject, counteracting the centre of gravity of the bra pad module, and/or correcting postural distortion due to imbalance between left and right sides of the mastectomized subject's upper torso post-mastectomy.

10. The customized module-wise posture correction bra of claim 1, wherein the one or more structural and functional elements further comprises detachable attachments comprising chest panel and pressure release shoulder strap for providing comfort for the mastectomized subject during wearing the bra and counteracting forces exerted on different parts of the mastectomized subject by the weight of the bra pad module or the bra through retraction.

11. The customized module-wise posture correction bra of claim 1, further comprising cups, underband, wings, bones, shoulder strap, and fastening means for closure.

12. The customized module-wise posture correction bra of claim 1, further comprising a means comprising a pocket, button, and/or hook and loop fastener, for securing the bra pad module to the bra.

13. The customized module-wise posture correction bra of claim 1, wherein the 3D printed bra pad is coated by a durable water repellent layer selected from the group consisting of silicon-based, wax-based, and plant-based polyfluorocarbon (PFC)-free materials, or any combination thereof.

14. A method for making the customized module-wise posture correction bra according to any of the preceding claims, the method comprising:
mapping breast contour of the existing breast or with reference to a database of women's breast anatomy information of a corresponding community;
determining size or volume of the existing breast or with reference to said database;
determining mass density of the existing breast or with reference to said database;
estimating size, volume, and mass density of the missing breast based on the size, volume, and mass density data of the existing breast or with reference to said database;
3D printing the bra pad based on the estimated size, volume and mass density of the missing breast;
making or selecting a weight insert corresponding to the mass density of the existing breast or with reference to said database;
assembling the bra pad and weight insert into the enclosure to form the bra pad module;
making a module-wise mastectomy bra; and
securing the bra pad module to the module-wise mastectomy bra and incorporating one or more of the structural and functional elements into the module-wise mastectomy bra according to the mastectomized subject's needs in order to obtain the customized module-wise posture correction bra.

15. The method of claim 14, wherein said mapping the breast contour and said determining the size or volume of the existing breast are both performed by 3D scanning.

16. The method of claim 14, wherein said determining the mass density of the existing breast or with reference to said database is performed by ultrasonography.

17. The method of claim 15, wherein said estimating the size, volume, and mass density of the missing breast is performed by subtracting a mirror image of the 3D scanned existing breast or with reference to said database from a mirror image of a 3D scanned existing breast covered with a standard bra.

18. The method of claim 16, wherein the mass density of the existing breast is determined by ultrasound scan data of fat and fibroglandular tissues in at least four quadrants at 12, 3, 6, and 9 o'clock positions, respectively, over the determined volume of the existing breast.

19. The method of claim 18, wherein the weight insert is made or selected with a mass density resembling the determined mass density of the existing breast by the following equation:
ρ_{B}=Aρ_{F}+(1−A)ρ_{G}
wherein the existing breast is assumed to be composed entirely of the fat and fibroglandular tissues, in which ρ_{F} denotes the mass density of fat tissue; ρ_{G} denotes the mass density of fibroglandular tissue; A denotes proportion of fat in the existing breast.

20. The method of claim 14, wherein the bra pad is 3D printed based on a random Voronoi approach to obtain a Voronoi-like 3D printed grid system, and the Voronoi-like 3D printed grid system is made of one or more elastic materials comprising thermoplastic elastomer (TPE), thermoplastic polyurethane (TPU), silicone, resin and/or viscoelastic gel.

21. The method of claim 14, wherein the weight insert is disposed in a space of the bra pad at about half to one-third of the determined height of the missing breast and close to chest wall of the mastectomized subject.

22. The method of claim 15 or 17, wherein said 3D scanning is performed by using a 3D scanner to scan the mastectomized subject's upper torso in a standing position A of ISO 20685-1:2018 in the presence or absence of the standard bra covering the existing and missing breasts.

23. The method of claim 14, further comprising coating on the bra pad with a durable water repellent layer selected from the group consisting of silicon-based, wax-based, and plant-based polyfluorocarbon (PFC)-free materials, or any combination thereof, prior to said assembling.
